(19) United States
(12) Patent Application Publication
(10) Pub. No.: US 2010/0028992 A1
(43) Pub. Date: Feb. 4, 2010

(54) Cell culture device

(75) Inventors: Tzu-Wei Wang, Taipei County (TW); Feng-Huei Lin, Taipei (TW)

Correspondence Address: BACON & THOMAS, PLLC, 625 SLATERS LANE, FOURTH FLOOR, ALEXANDRIA, VA, 22314-1176, US

(73) Assignee: National Taiwan University, Taipei (TW)

(21) Appl. No.: 12/314,631

(22) Filed: Dec. 15, 2008

(30) Foreign Application Priority Data
Jul. 30, 2008 (TW) 097128727

(51) Int. Cl. C12M 1/02; C12M 3/00

(52) U.S. Cl. 435/302.1; 435/289.1; 435/305.3; 435/305.4

(57) ABSTRACT

The present invention discloses a cell culture device, including a container, and a carrying assembly; the container has an opening and holds the cell culture medium, and the carrying assembly is immersed in the cell culture solution from the opening of the container. The present invention also discloses a carrying assembly used for cell culture, including a cover, a supporter, and a vessel, wherein the cover and the vessel are connected by the supporter.

### BACKGROUND OF THE INVENTION

1. Field of the Invention

The invention relates to a cell culture device, particularly to a cell culture device used for culturing the attached cells.

2. Description of the Prior Art

With the development of biotechnology, no matter in the gene screening or new medicine development, the cell culture technology is often used. However, many kinds of cell are still difficult to be cultured, and the space and cost required to culture a large number of cells are still very limited and expensive.

Traditionally, cell culture Petri-dish or cell culture flask has to be used to culture the attached cells. Each culture container only can contain a limited number of cells. If a large number of cells are going to be cultured, a certain space is required to pile these culture containers, which will increase the difficulty of handling. Besides, these containers are disposal containers, which cost very expensive and will become the burden of the environment. Most importantly, this kind of culture method is limited to 2D culture, which has big difference compared to 3D culture environment in actual tissue growth. The phenotype and morphology of cells are easy to be changed in former culture environment, and these cells tend to differentiate to fibroblast-like cells. The latter biomimics the actual growth of organism, which can maintain the cell phenotype and function effectively and can form three-dimensional extracellular matrix structure much closer to the actual situation in tissue growth.

If a large number of cells are going to be cultured, though the spinner flask or roller wall vessel reactor can be applied, these culture methods are mostly suitable for culturing non-attached cells, which mean they can only be used to culture suspended cells and are not suitable for all kinds of cells.

In order to culture the attached cells effectively, Taiwan Patent No. 1238851 discloses a bioreactor with an eggshell and a fluid mixing device. The bioreactor uses the inside wall structure of eggshell, which includes a porous or net part. Although this bioreactor can be used to culture small amount of cells, it uses the eggshell as the container and needs additional fluid mixing device. The operation is also not convenient in general laboratories.

Furthermore, Taiwan Patent No. 1222997 provides an in-vitro tissue culture method. The general spinner flask is used to culture the tissue. After the tissue has been treated, it is placed in a hollow porous carrier, and incubated in the spinner flask for culturing. However, the porous carrier is a polymer material with specific biological absorbability, which is mainly used for the culture and transplantation of cartilage cells and is unsuitable to be used for other kinds of cells.

U.S. Pat. No. 6,991,933 also discloses the application of another kind of cell culture device. A plurality of spiral arms and jigs are set to support the carrier of cell culture, in order to culture a large number of cells. Its structure is comparatively complicated, and the cost of setup is relatively high.

Therefore, in order to solve the above inconvenience and problems of culturing attached cells in large number, the present invention provides a cell culture device to culture the attached cells in more effective and economical way. The invention is described briefly as follows.

### SUMMARY OF THE INVENTION

The present invention relates to a cell culture device, particularly to the modification of existing cell culture device, in order to culture the attached cells. The present invention also provides a carrying assembly used for cell culture.

One aspect of the present invention is to provide a cell culture device, which includes a container and a carrying assembly. The container has an opening and holds cell culture solution. The carrying assembly is immersed in the cell culture medium from the opening of the container.

In a preferred embodiment, the opening further has a detachably corresponding cap.

In another preferred embodiment, the carrying assembly includes a cover, a supporter, and a vessel.

Preferably, the supporter is connected to the cover at one end and connected to the vessel at the other end.

In another preferred embodiment, wherein the vessel is a basket with single chamber or multiple chambers, or a cartridge.

Most preferably, the vessel holds a carrier for cell culture.

In another preferred embodiment, the vessel has porous or meshed structure to neutralize the shear flow of cell culture media.

In another preferred embodiment, the vessel is immersed in the cell culture solution.

In another preferred embodiment, the cell culture device of the present invention further includes a spin device.

Preferably, the spin device is a magnetic stirrer device, a mechanical spin device, or a fluidized spin device.

Most preferably, the magnetic spin device includes a stirrer at the bottom of the container and a magnetic power supply outside the container.

In another preferred embodiment, the container further has one opening at both sides of the container respectively.

Another aspect of the present invention is to provide a cell culture device, which includes a container, a carrying assembly, and a spin device. The container has an opening and holds cell culture medium. The carrying assembly is immersed in the cell culture solution from the opening of the container. The opening has a detachably corresponding cap.

In a preferred embodiment, the carrying assembly includes a cover, a supporter, and a vessel, wherein the cover and the vessel are connected to each end of the supporter respectively.

Preferably, the vessel is a basket with single chamber or multiple chambers, or a cartridge, to hold a carrier for cell culture.

In another preferred embodiment, the vessel has porous or meshed structure to neutralize the shear flow of cell culture solution.

In another preferred embodiment, the spin device is a magnetic stirrer device, a mechanical spin device, or a fluidized spin device.

Another aspect of the present invention is to provide a carrying assembly for cell culture, which includes a cover, a supporter, and a vessel; wherein the cover and the vessel are connected by the supporter.

In a preferred embodiment, the vessel, which holds a carrier for cell culture, is a basket with single chamber or multiple chambers, or a cartridge.

More preferably, the carrier for cell culture is a film, a plate, or a sponge composed of polymers or biocompatible materials, for example, films and plates composed of polymers (e.g., polylactic acid, polyglycolic acid, chitosan, hyaluronic acid, and glycosaminoglycan), and sponge (such as, collagen, gelatin, and artificial bone) with biological absorbability.

In another preferred embodiment, the vessel has porous, meshed or lattice structure.

The carrying assembly of the invention consists of a supporter, a vessel and a cover. The supporter can be plastics, glass or other materials, preferably stainless steel, wherein the both ends are connected to the cover and vessel, respectively. The cover is disposable. The vessel is a basket with single chamber or multiple chambers, preferably a cartridge, which depends on the species and number of cells to be cultured. The material of the vessel can also be treated by high-pressure and high-temperature autoclave sterilization. The preferred example is disposable, in order to reduce the possibility of bacteria contamination in the cell culture process. The surface of vessel has a lot of pores, and the preferred embodiment is a meshed or lattice structure, in order to neutralize the shear flow of cell culture solution in the culture process. The carrier is put within the vessel, which can avoid the interference of cell growth on the surface of carrier due to the excess disturbance of culture fluid.

The cell culture device of the invention can be used to culture attached cells, particularly suitable to the cells which are hard to be cultured, such as adult stem cells with multipotency, including but not limited to the culture of bone marrow-derived mesenchymal stem cells, or the primary culture of cells which are easily to loose their cell phenotype during cultivation, such as chondrocytes, endothelial cells, osteoblasts, or dermal fibroblasts.

The cell culture device of the invention improves the existing cell culture container, in order to utilize a small amount of cell culture media to create and keep cell culture, and reduce the production cost.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 shows the schematic diagram of common cell culture bottle glassware.

FIG. 2 shows the schematic diagram of cell culture carrier of the invention.

FIG. 3 shows the schematic diagram of cell culture device of the invention.

FIG. 4 shows the growth curve of cells cultured by the cell culture device of the invention.

FIG. 5A and FIG. 5B show the comparison for cell growth curve among the cell culture device of the invention and traditional cell culture devices.

### DESCRIPTION OF THE PREFERRED EMBODIMENT

The cell culture device of the invention can be fully understood by the following embodiments. The person skilled in the art can complete it accordingly, but the implementation type of the invention is not limited to the following embodiments.

### Embodiment 1

### Setup of Cell Culture Device

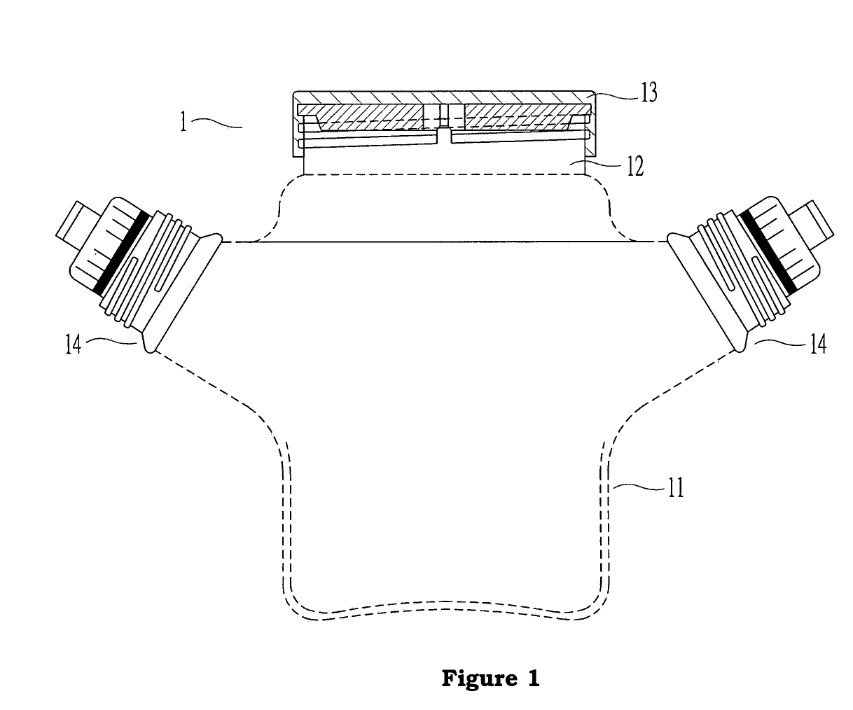

Please refer to FIG. 1, which shows the schematic diagram of a commercialized 125 ml cell culture glassware. The cell culture glassware 1 has a bottle body 11 to contain the cell culture medium. There are an opening 12 and a corresponding cap 13 above it. In addition, this cell culture glassware has the openings 14 at both sides of bottle body. These two openings will not be closed completely for the purpose of air exchange. When it is required to refresh the culture medium, the side openings are used for the replacement. Some kinds of cell culture glassware do not have side openings; thus, the cap will not be tightened totally at this moment for the air exchange.

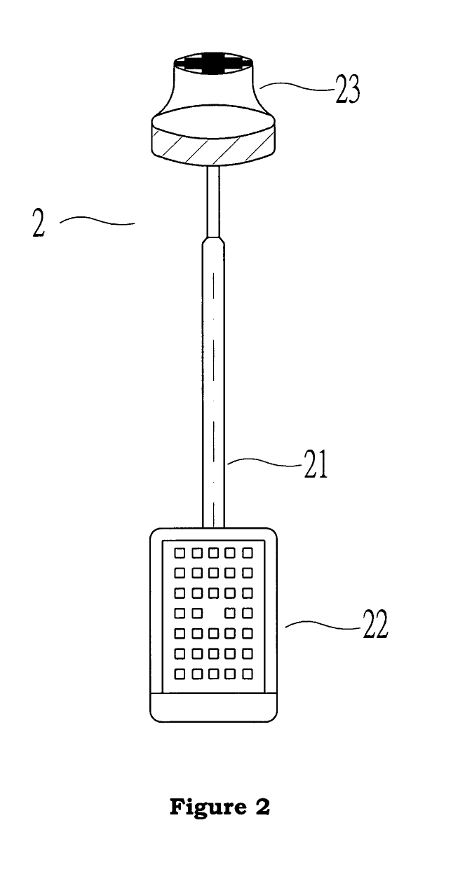

Please refer to FIG. 2, which shows the schematic diagram of carrying assembly according to the present invention. The carrying assembly 2 is mainly composed of the supporter 21, vessel 22 and cover 23. The supporter 21 is stainless steel, and the cover 23 and vessel 22 are connected to each end of the supporter 21, respectively. The vessel 22 is a basket with single chamber or multiple chambers, and the preferred one is a cartridge. The vessel 22 may be disposable, in order to reduce the possibility of contamination in the cell culture procedure. The surface of vessel 22 has a lot of pores, and the preferred embodiment is meshed or lattice structure.

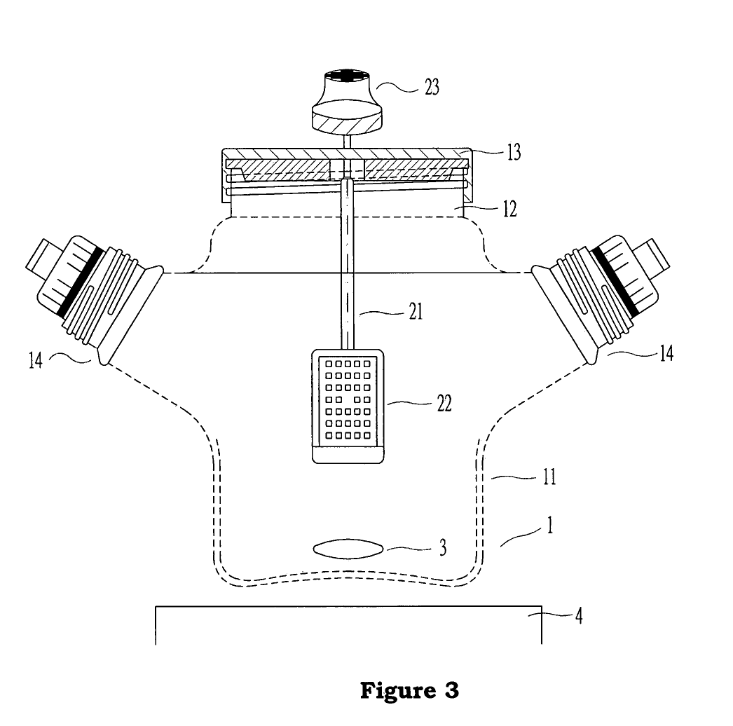

Please refer to FIG. 3, which shows the schematic diagram of cell culture device of the invention. The cell culture bottle 1 and carrying assembly 2 are combined to form the cell culture device of the present invention. In order to set up the carrying assembly 2 properly, a hole is punched in the middle of cap 13, and the supporter 21 is passed through it. After the vessel 22 and cover 23 are assembled, the cap 13 with carrying assembly 2 are set back to cell culture bottle 1 to complete the setup of cell culture device according to the present invention.

### Embodiment 2

### Cell Culture Experiment

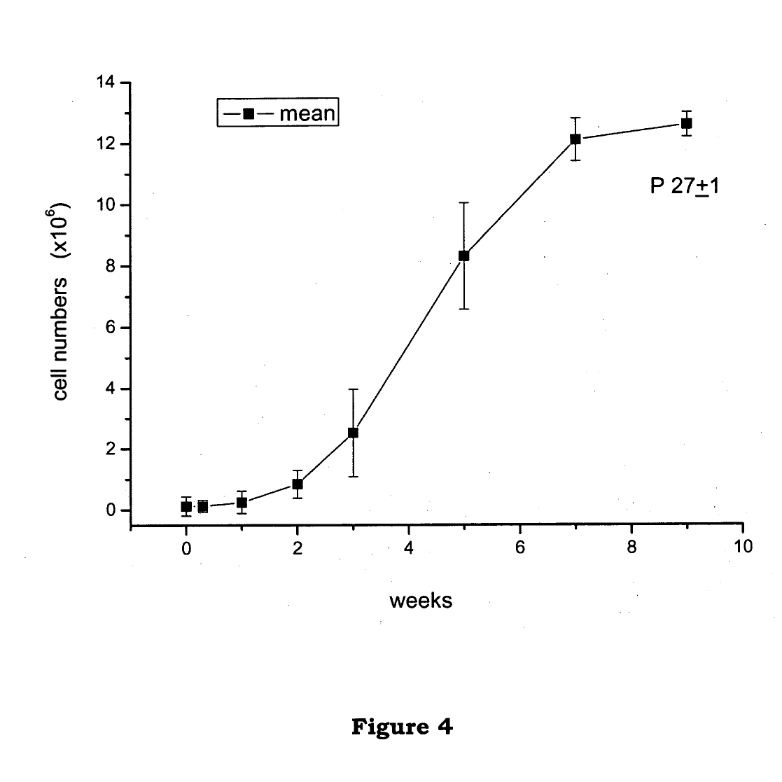

The mesenchymal stem cells (MSC) are cultured in cell culture dish, the cell culture device of the invention, and the rotating wall vessel bioreactor, respectively. According to the method specified in Taiwan Patent Application No. 096,126,468, one can prepare a porous polymer carrier for the cell attachment and growth. After the cell/polymer carrier is placed in the vessel 22, 100 ml cell culture medium is added into the cell culture bottle and then assembles the cell culture device. It is cultured in a humidified incubator at 37 □, 5% CO₂. The rotational speed of stirrer is adjusted to 60 r.p.m., and the cell culture medium is refreshed every 2 to 3 days. The result of cell growth is shown in FIG. 4. The cell number is 0.12±0.02×10⁶ cells at day 3, and increase to 0.25±0.06×10⁶ cells at day 7. Cells continue to grow in the form of exponential order. The cell number is growing up to 12.10±0.72×10⁶ in the seventh week, which is more than 100 times of initial number. The cells still can be cultured until the ninth week in the cell culture device of the invention.

In addition, when the rotating wall vessel bioreactor is used, the cell/polymer carrier is placed in the rotating bottle (Synthecone Company, USA), and the rotational speed of stirrer is 30 r.p.m.

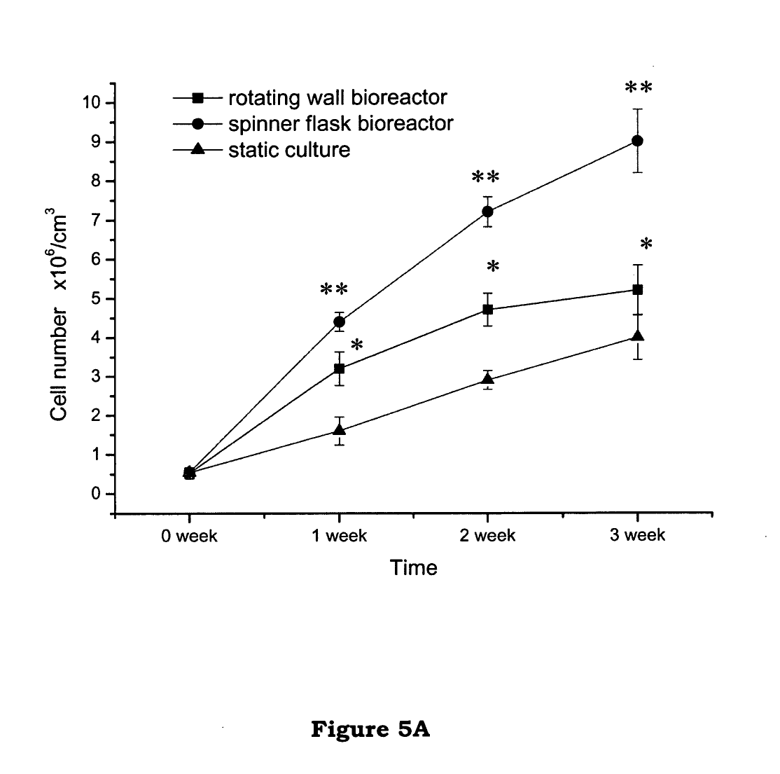

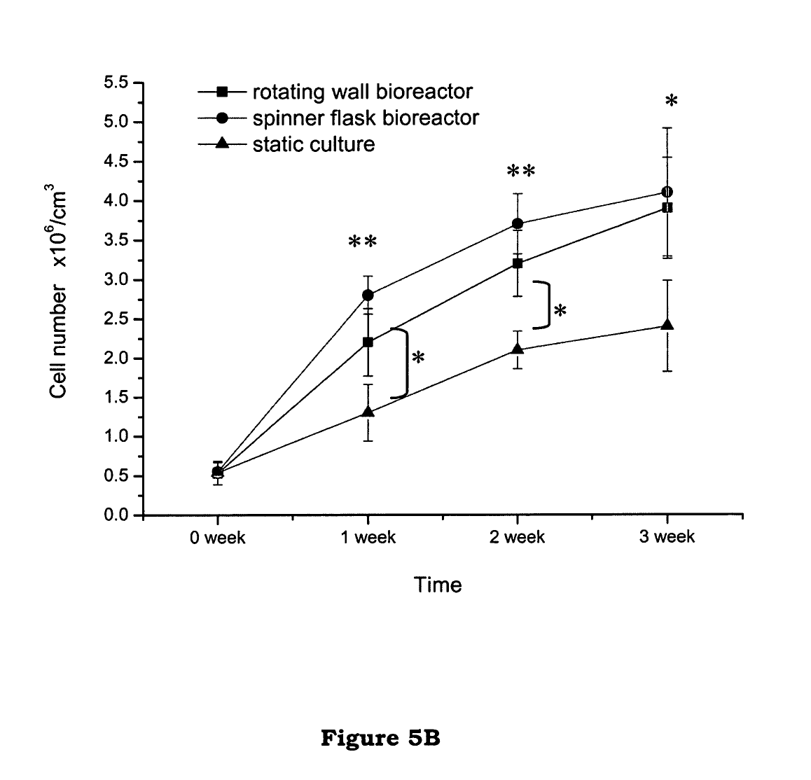

In order to verify the differentiation ability of stem cells, the stem cells was induced into osteoblasts or chondrocytes. The induction solution shall be replaced every 3 to 4 days. It is found that the result of stem cell differentiation in the invention is better than that in other two traditional methods (as shown in FIGS. 5A and 5B).

It is understood that various other modifications will be apparent to and can be readily made by those skilled in the art without departing from the scope and spirit of this invention. Accordingly, it is not intended that the scope of the claims appended here to be limited to the description as set forth herein, but rather that the claims be construed as encompassing all the features of patentable novelty that reside in the invention, including all features that would be treated as equivalents thereof by those skilled in the art to which this invention pertains.

What is claimed is:

1. A cell culture device, comprising:
a container having an opening and holding cell culture medium;
and a carrying assembly immersed in the cell culture solution from the opening of the container.

2. The device according to claim 1, wherein the opening further has a detachably corresponding cap.

3. The device according to claim 1, wherein the carrying assembly comprises a cover, a supporter, and a vessel.

4. The device according to claim 3, wherein the supporter is connected to the cover at one end and connected to the vessel at the other end.

5. The device according to claim 3, wherein the vessel is a basket with single chamber, multiple chambers, and a cartridge.

6. The device according to claim 5, wherein the vessel holds a carrier for cell culture.

7. The device according to claim 5, wherein the vessel has porous and meshed structure to neutralize the shear flow of cell culture solution.

8. The device according to claim 3, wherein the vessel is immersed in the cell culture solution.

9. The device according to claim 1 further comprising a spin device.

10. The device according to claim 9, wherein the spin device is a magnetic stirrer, a mechanical spin device, and a fluidized spin device.

11. The device according to claim 10, wherein the magnetic spin device comprises a stirrer at the bottom of the container and a magnetic power supply outside the container.

12. The device according to claim 1, wherein the container further has one opening at both sides of the container respectively.

13. A cell culture device, comprising:
a container having an opening and hold a cell culture solution; a carrying assembly immersed in the cell culture solution from the opening of the container; and a spin device; wherein the opening has a detachably corresponding cap.

14. The device according to claim 13, wherein the carrying assembly comprises a cover, a supporter, and a vessel, wherein the cover and the vessel are connected to each end of the supporter respectively.

15. The device according to claim 14, wherein the vessel is a basket with single chamber and multiple chambers, and a cartridge, to hold a carrier for cell culture.

16. The device according to claim 14, wherein the vessel has porous and meshed structure to neutralize the shear flow of cell culture solution.

17. The device according to claim 13, wherein the spin device is a magnetic stirrer device, a mechanical spin device, and a fluidized spin device.

18. A carrying assembly for cell culture, comprising a cover, a supporter, and a vessel, wherein the cover and the vessel are connected by the supporter.

19. The carrying assembly according to claim 18, wherein the vessel is a basket with single chamber or multiple chambers, and a cartridge to hold a carrier for cell culture.

20. The carrying assembly according to claim 19, wherein the carrier for cell culture is a film, a plate, and a sponge composed of polymers or biocompatible materials.
